(19) United States
(12) Patent Application Publication
(10) Pub. No.: US 2012/0284046 A1
(43) Pub. Date: Nov. 8, 2012

(54) Computer program product for reporting a result of an evaluation of a sample after queuing the result for transmission

(75) Inventors: Michael Hartmann Baym, Cambridge, MA (US); Roderick A. Hyde, Redmond, WA (US); Jordin T. Kare, Seattle, WA (US); Erez Lieberman, Cambridge, MA (US); Elizabeth A. Sweeney, Seattle, WA (US); Lowell L. Wood, JR., Bellevue, WA (US)

(73) Assignee: Searete LLC

(21) Appl. No.: 13/068,291

(22) Filed: May 6, 2011

(51) Int. Cl. G06Q 50/00; C12Q 1/68; C12Q 1/04; C12Q 1/70

(52) U.S. Cl. 705/3; 435/5; 435/6.15; 435/34

(57) ABSTRACT

A computer program product includes a recordable-type signal bearing medium bearing computer usable code configured for causing an automatic evaluation, remotely from a health care facility, of a biological sample acquired from a subject for a presence or an absence of at least one pathogen in the biological sample; computer usable code configured for electronically queuing a first result of the evaluation for transmission to an off-site entity; and computer usable code configured for reporting a second result of the evaluation to the subject after queuing the first result of the evaluation for transmission.

### SUMMARY

In one aspect, a method includes, but is not limited to, automatically evaluating, remotely from a health care facility, a biological sample acquired from a subject for a presence or an absence of at least one pathogen in the biological sample; electronically queuing a first result of the evaluation for transmission to an off-site entity; and reporting a second result of the evaluation to the subject after queuing the first result of the evaluation for transmission. In addition to the foregoing, other method aspects are described in the claims, drawings, and text forming a part of the present disclosure.

In one or more various aspects, related systems include but are not limited to circuitry and/or programming for effecting the herein-referenced method aspects; the circuitry and/or programming can be virtually any combination of hardware, software, and/or firmware configured to effect the herein-referenced method aspects depending upon the design choices of the system designer.

In one aspect, a system includes, but is not limited to, an evaluation module for automatically evaluating, remotely from a health care facility, a biological sample acquired from a subject for a presence or an absence of at least one pathogen in the biological sample; an electronic memory for queuing a first result of the evaluation for transmission to an off-site entity; and a reporting module for reporting a second result of the evaluation to the subject after queuing the first result of the evaluation for transmission. In addition to the foregoing, other system aspects are described in the claims, drawings, and text forming a part of the present disclosure.

In one aspect, a device includes, but is not limited to, an evaluation module for automatically evaluating, remotely from a health care facility, a biological sample acquired from a subject for a presence or an absence of at least one pathogen in the biological sample; an electronic memory coupled with the evaluation module for queuing a first result of the evaluation for transmission to an off-site entity; and a reporting module coupled with the evaluation module for reporting a second result of the evaluation to the subject after queuing the first result of the evaluation for transmission. In addition to the foregoing, other system aspects are described in the claims, drawings, and text forming a part of the present disclosure.

In one aspect, a computer program product includes, but is not limited to, a recordable-type, non-transitory, signal-bearing medium bearing computer usable code configured for causing an automatic evaluation, remotely from a health care facility, of a biological sample acquired from a subject for a presence or an absence of at least one pathogen in the biological sample; computer usable code configured for electronically queuing a first result of the evaluation for transmission to an off-site entity; and computer usable code configured for reporting a second result of the evaluation to the subject after queuing the first result of the evaluation for transmission.

In addition to the foregoing, various other method and/or system and/or program product aspects are set forth and described in the teachings such as text (e.g., claims and/or detailed description) and/or drawings of the present disclosure.

The foregoing is a summary and thus may contain simplifications, generalizations, inclusions, and/or omissions of detail; consequently, those skilled in the art will appreciate that the summary is illustrative only and is NOT intended to be in any way limiting. Other aspects, features, and advantages of the devices and/or processes and/or other subject matter described herein will become apparent in the teachings set forth herein.

### BRIEF DESCRIPTION OF THE FIGURES

FIG. 1 is a schematic.

FIG. 2 illustrates an operational flow representing example operations.

FIG. 3 illustrates an alternative embodiment of the operational flow of FIG. 2.

FIG. 4 illustrates an alternative embodiment of the operational flow of FIG. 2.

FIG. 5 illustrates an operational flow representing example operations.

FIG. 6 illustrates an operational flow representing example operations.

FIG. 7 illustrates an operational flow representing example operations.

FIG. 8 illustrates an operational flow representing example operations.

FIG. 9 illustrates an operational flow representing example operations.

FIG. 10 illustrates an operational flow representing example operations.

FIG. 11 illustrates an alternative embodiment of the operational flow of FIG. 8.

FIG. 12 illustrates a computer program product related to reporting a result of an evaluation of a sample after queuing the result for transmission.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments can be utilized, and other changes can be made, without departing from the spirit or scope of the subject matter presented here.

The state of the art has progressed to the point where there is little distinction left between hardware, software, and/or firmware implementations of aspects of systems; the use of hardware, software, and/or firmware is generally (but not always, in that in certain contexts the choice between hardware and software can become significant) a design choice representing cost vs. efficiency tradeoffs. There are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer can opt for a mainly hardware and/or firmware vehicle; alternatively, if flexibility is paramount, the implementer can opt for a mainly software implementation; or, yet again alternatively, the implementer can opt for some combination of hardware, software, and/or firmware. Hence, there are several possible vehicles by which the processes and/or devices and/or other technologies described herein can be effected, none of which is inherently superior to the other in that any vehicle to be utilized is a choice dependent upon the context in which the vehicle will be deployed and the specific concerns (e.g., speed, flexibility, or predictability) of the implementer, any of which can vary. Optical aspects of implementations will typically employ optically-oriented hardware, software, and or firmware.

In some implementations described herein, logic and similar implementations can include software or other control structures. Electronic circuitry, for example, can have one or more paths of electrical current constructed and arranged to implement various functions as described herein. In some implementations, one or more media can be configured to bear a device-detectable implementation when such media hold or transmit a device detectable instructions operable to perform as described herein. In some variants, for example, implementations can include an update or modification of existing software or firmware, or of gate arrays or programmable hardware, such as by performing a reception of or a transmission of one or more instructions in relation to one or more operations described herein. Alternatively or additionally, in some variants, an implementation can include special-purpose hardware, software, firmware components, and/or general-purpose components executing or otherwise invoking special-purpose components. Specifications or other implementations can be transmitted by one or more instances of tangible transmission media as described herein, optionally by packet transmission or otherwise by passing through distributed media at various times.

Alternatively or additionally, implementations can include executing a special-purpose instruction sequence or invoking circuitry for enabling, triggering, coordinating, requesting, or otherwise causing one or more occurrences of virtually any functional operations described herein. In some variants, operational or other logical descriptions herein can be expressed as source code and compiled or otherwise invoked as an executable instruction sequence. In some contexts, for example, implementations can be provided, in whole or in part, by source code, such as C++, or other code sequences. In other implementations, source or other code implementation, using commercially available and/or techniques in the art, can be compiled/implemented/translated/converted into a high-level descriptor language (e.g., initially implementing described technologies in C or C++ programming language and thereafter converting the programming language implementation into a logic-synthesizable language implementation, a hardware description language implementation, a hardware design simulation implementation, and/or other such similar mode(s) of expression). For example, some or all of a logical expression (e.g., computer programming language implementation) can be manifested as a Verilog-type hardware description (e.g., via Hardware Description Language (HDL) and/or Very High Speed Integrated Circuit Hardware Descriptor Language (VHDL)) or other circuitry model which can then be used to create a physical implementation having hardware (e.g., an Application Specific Integrated Circuit). Those skilled in the art will recognize how to obtain, configure, and optimize suitable transmission or computational elements, material supplies, actuators, or other structures in light of these teachings.

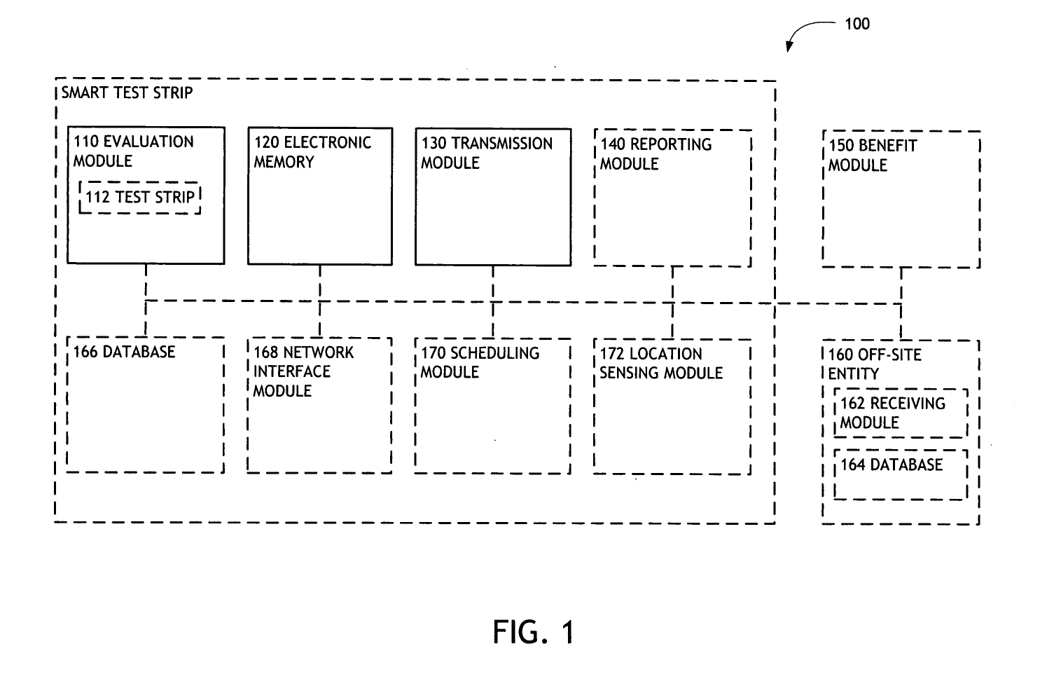

Referring now to FIG. 1, a system for the evaluation and reporting of a biological sample is described in accordance with the present disclosure. The system includes an evaluation module 110, an electronic memory 120, a transmission module 130, a reporting module 140, or, optionally, a benefit module 150. In an implementation, the evaluation module 110, the electronic memory 120, the transmission module 130, and the reporting module 140 are included as a unitary system 100. In an implementation, the evaluation module 110 is provided as a single device, and the electronic memory 120, the transmission module 130, and the reporting module 140 are provided as another device. In an implementation, the electronic memory 120 and the transmission module 130 are included as a single device, and the reporting module 140 is included as another device. A non-exhaustive list of devices that may be utilized with the present disclosure include a personal computer, a laptop computer, a palmtop computer, a Personal Digital Assistant (PDA), a mobile telephone, or an image capture device, such as a digital camera. It should be noted that this list is provided by way of example only, and other various devices may be utilized with the present disclosure. Further, different devices including different combinations of functionalities may be utilized to fulfill the various functionalities described for the evaluation module 110, the electronic memory 120, the transmission module 130, the reporting module 140, or the benefit module 150. For instance, the system 100 includes an evaluation module 110, an electronic memory 120, a transmission module 130, a reporting module 140, and a benefit module 150.

The evaluation module 110 included with the system 100 is for automatically evaluating the biological sample to determine whether one or more pathogens are present within the subject. For instance, the one or more pathogens can include one or more of a virus (such as Human Immunodeficiency Virus (HIV)), a parasite, a bacterium, a fungus, a toxin, or a toxin-producing pathogen. Additionally, the one or more pathogens can include one or more of a flu virus, such as an influenza A virus, an influenza A virus subtype, an influenza B virus, an influenza C virus, or the like. For example, the evaluation module 110 can detect, in the biological sample, the presence or absence of a pathogen. As used, herein, the term “pathogen” includes the pathogen itself or one or more indicators of the presence of pathogen in the subject. Such indicators of pathogen in the subject include an antigen from the pathogen, cognate to the pathogen, an antibody immunoreactive with the pathogen, a chemical, a toxin, a nucleic acid, a Pathogen-Associated Molecular Pattern (PAMP), or the like associated with the presence of the pathogen. Nucleic acids may be obtained from or found in DNA, RNA, or polynucleotide fragments of a pathogen. For example, a particular DNA sequence may be associated with a pathogen. In another instance, an unmethylated nucleic acid (CpG) may be associated with a bacterial pathogen. Further, double stranded RNA may be associated with a viral pathogen. PAMPs may include flagellin of bacterial flagella, peptidoglycans of Gram-positive bacteria, lipopolysaccharide (LPS or endotoxin) of Gram-negative bacteria, Lipoteichoic acid (LTA) Gram-positive bacteria, double-stranded RNA, or unmethylated DNA. Additionally, the evaluation module 110 can detect the presence or absence of one or more pathogens directly in the sample.

A subject provides a biological sample for evaluation by the evaluation module 110. The sample can be acquired remotely from a health care facility. Further, the evaluation module 110 provides the automatic evaluation of the biological sample remotely from a health care facility. In the context of the present disclosure, a “health care facility” includes any location where the evaluation of biological samples is performed, or where the subject would be exposed to biological pathogens from persons or environmental factors outside those normally found in a residential-type environment not associated with group health care activities. A non-exhaustive list of health care facilities would include hospitals, outpatient care clinics, emergency care clinics, and health screening centers and diagnostic laboratories. In an embodiment, the sample includes one or more of a biological sample, such as fluid, a cell, or tissue obtained via, for example, a nasal swab, an oral swab, a thoracic swab, a nasal aspirate, an oral aspirate, a thoracic aspirate, a nasal wash, an oral wash, a thoracic wash, a tissue/skin scrape, phlegm, sputum, saliva, urine, blood, or the like. In embodiments, the evaluation module 110 can include one or more of a test strip, a “lab-on-a-chip” device, an assay (e.g., a Micro-Electro-Mechanical Systems/microelectronic and microelectromechanical systems (MEMS) bio-assay device), a lateral flow test strip, a colorimetric test strip, a lateral flow colorimetric test strip, a transdermal testing device, a lancet, or the like for determining the presence or the absence of one or more pathogens in a sample.

In an embodiment, a colorimetric test strip can be aptamer based. A nucleic acid aptamer that is a cognate to a target analyte is conjugated to a strip or card. Upon binding, the test strip changes color which is easily detected. Other embodiments include colorimetric test strips using antibody-conjugates, such as the Triage Parasite Panel, developed by Biosite Diagnostics, San Diego, Calif. The Triage® strip is a single immuno-chromatographic (IC) strip coated with monoclonal antibodies specific for 29-kDa surface antigen (E. histolytica-E. dispar), alpha-1-giardin (G. lamblia), and protein disulfide isomerase (C. parvum). A positive reaction in the Triage kit is identified by a qualitative colorimetric reaction when the antibody conjugate (alkaline phosphatase) reacts with the substrate (indoxyl phosphate), resulting in an easily detectable dark blue-purple line on the IC strip.

In an embodiment, an evaluation module 110 includes a lateral flow test strip. The lateral flow test strip generally includes a sample pad—an absorbent pad onto which the test sample is applied, a conjugate or reagent pad, containing antibodies specific to the pathogen conjugated to colored particles (typically, these can include gold particles, or latex microspheres). Also included is a reaction membrane, typically a hydrophobic nitrocellulose or cellulose acetate membrane onto which anti-pathogen antibodies are immobilized in a line across the membrane as a capture zone or test line (a control zone may also be present, containing antibodies specific for the conjugate antibodies); a wick or waste reservoir—a further absorbent pad designed to draw the sample across the reaction membrane by capillary action and collect it. The components of the lateral flow test strip are fixed to an inert backing material and may be presented in a simple dipstick format or within a plastic casing with a sample port and reaction window showing the capture and control zones.

An example of a lateral flow test strip includes double antibody sandwich assays. The biological sample migrates through the conjugate pad where any pathogen present will bind to the conjugate. The pathogen:conjugate complex then continues to migrate across the membrane until it reaches the capture zone where the pathogen:conjugate complex will bind to the immobilized antibodies producing a visible, and detectable, line on the membrane. The sample then migrates further along the strip until it reaches the control zone, where excess conjugate will bind and produce a second visible, and detectable, line on the membrane. This control line indicates that the sample has migrated across the membrane as intended. Two clear lines on the membrane is a positive result. A single line in the control zone is a negative result. Double antibody sandwich assays are suitable for larger analytes, such as, without limitation, bacterial pathogens and viruses, with multiple antigenic sites.

Competitive assays utilize a conjugate pad that contains antibodies that are already bound to the pathogen of interest. If the pathogen is present in the biological sample, it will therefore not bind with the conjugate and will remain unlabelled. As the sample migrates along the membrane and reaches the capture zone an excess of unlabelled pathogen will bind to the immobilized antibodies and block the capture of the conjugate, so that no visible line is produced. The unbound conjugate will then bind to the antibodies in the control zone producing a visible control line. A single control line on the membrane is a positive result. Two visible lines in the capture and control zones is a negative result. However, if an excess of unlabelled pathogen is not present, a weak line may be produced in the capture zone, indicating an inconclusive result. Competitive assays are most suitable for testing for small molecules, such as mycotoxins, unable to bind to more than one antibody simultaneously. There are a number of conventional variations on lateral flow technology, which are included within the scope of the invention. The capture zone on the membrane may contain immobilized antigens or enzymes, depending on the target analyte, rather than antibodies. In an embodiment, multiple capture zones are used to create a multiplex test strip. For example, commercial test strips able to detect both EHEC Shiga toxins ST1 and ST2 separately in the same sample have been developed.

Lateral flow immunoassays generally produce a result within 15 minutes. It is possible to obtain a degree of quantification by measuring the amount of conjugate bound to the capture zone. This can be done using a dedicated reader to measure the intensity of the colored test line. For example, Neogen Corporation has developed the Accuscan™ lateral flow reader for use with its range of Reveal® assay kits and Charm Sciences also supplies a reader for its Rosa® range of mycotoxin test strips. Other techniques, such as use of fluorescent dye labelled conjugates, can improve the quantitative potential of lateral flow assays.

An embodiment includes an evaluation module 110 including lab-on-a-chip devices. See, for example, Schulze, et al., J. Biophotonics. 2009 April; 2 (4):199-211, which is incorporated herein by reference. An embodiment can include peptide nanotube sensors such as those described in de la Rica, et al., Angewandte Chemie International Edition, Volume 47, Issue 50, 9752-9755, December 2008; which is incorporated herein by reference. Such peptide nanotube sensors utilize a pair of gold electrodes bridged with antibody-coated peptide nanotubes. The nanotubes concentrate the pathogen on their surface by molecular recognition between the antibody and the pathogen. The nanotubes fit within the electric field line distribution to enable the extremely sensitive impedimetric detection of pathogen.

An embodiment includes an evaluation module 110 including a lab-on-a-chip device that uses light scattering detection of immunoagglutination. See, for example, “Lab-on-a-Chip for Field Escherichia coli Assays: Long-Term Stability of Reagents and Automatic Sampling System,” Journal of the Association for Laboratory Automation, Volume 15, Issue 3, Pages 216-223; which is incorporated herein by reference.

An embodiment includes an evaluation module 110 including one or more sensors that include a biological molecule capture layer. The biological molecule capture layer can include an array of different binding molecules that specifically bind one or more target molecules (pathogens). For example, in an embodiment, the one or more sensors include an array of micro-regions modified to capture pathogens. In an embodiment, the one or more sensors include one or more surface plasmon resonance sensors for detecting captured pathogens. For example, surface-plasmon-resonance-based-sensors detect pathogens suspended in a fluid by reflecting light off thin metal films in contact with the fluid. Adsorbing molecules cause changes in the local index of refraction, resulting in detectable changes in the resonance conditions of the surface plasmon waves.

In an embodiment, the evaluation module 110 can include a test strip having a chemical sensor, a test assay, or any other type of instrumentation capable of determining the presence or absence of a pathogen, such as instrumentation for performing a biochemical assay. In an embodiment, a user can provide a biological sample to a collector for evaluation by the evaluation module 110. In an embodiment, the evaluation module 110 is separate from the collector. For example, a user can provide a biological sample to the collector (e.g., a cup for collecting urine); the user can then insert the test strip into the biological sample for evaluation. In an embodiment, the collector can be integrated in the evaluation module 110. For example, the evaluation module 110 can include a home health appliance. A user can provide a biological sample to the collector and insert the collector into the home health appliance for evaluation.

In an embodiment, the evaluation module 110 can include a unique identifier. The unique identifier can include one or more of a bar code, an RFID tag, or a wireless tag. In embodiments, the unique identifier can indicate one or more of a lot number, a group assignment, a location of obtainment, a location of reporting, a date of obtainment, a date of reporting, a time elapsed between a date of obtainment and a date of reporting, or an evaluation module 110 type.

In an embodiment, the evaluation module 110 can store information associated with the presence or the absence of the at least one pathogen in the biological sample in the electronic memory 120 for storage or transmission to an off-site entity 160. The information associated with the presence or the absence of the at least one pathogen can be stored in an encoded format. The off-site entity 160 can be one or more of a medical care provider, a public health agency, a government agency, or an insurance agency. For instance, and without limitation, the off-site entity 160 can be at least one of the Centers for Disease Control and Prevention (CDC), the Federal Emergency Management Agency (FEMA), or a federal agency in the Department of Health and Human Services operated by the United States government.

In an embodiment, the information associated with the presence or the absence of the at least one pathogen may be collated by the off-site entity 160 in surveillance of infectious disease transmission, including surveillance of emerging infections diseases (EID). Such information may be utilized for monitoring diseases in populations and procuring information on disease outbreaks and epidemics. EIDs may include diseases caused by newly identified microorganisms or newly identified strains of known microorganisms, new infections resulting from a change or evolution of an existing organism, a known infection which spreads to a new geographic area or population, a newly recognized infection in an area undergoing ecologic transformation, or pre-existing and recognized infections reemerging due to drug resistance or a deterioration in public health. Further, the off-site entity 160 may monitor adverse synergetic interaction among emerging diseases and interaction with other infectious and non-infectious conditions that lead to the development of novel syndemics, where interaction between diseases may exacerbate health effects of the diseases.

The evaluation module 110 may provide one or more test results, such as a first result indicating the presence or absence of a pathogen in the sample. The first result of the evaluation can be communicated, or reported, to the subject or user. The first result can be communicated in full to the subject or user, or communicated in a limited fashion. Further, in an embodiment, the first result can be communicated utilizing obfuscation to prevent communication to non-users or non-subjects. In an embodiment, the obfuscated format can include one or more of an encoded format, an alphanumeric code, an image, or a spoken word format. Further, the information associated with the presence or the absence of the at least one pathogen in the sample can be stored in the electronic memory 120 as an obfuscated first result of the evaluation. In an embodiment, the first result of the evaluation can be queued in the electronic memory 120 for subsequent transmission to an off-site entity 160.

In an embodiment, the first result of the evaluation indicating the presence or the absence of the pathogen in the sample can be determined with respect to one or more of a value, a threshold, or a multiplicity of thresholds. For example, the presence of a pathogen may be determined by a measurement of an indicator exceeding a threshold value. Alternatively, the presence of a pathogen may be determined by measuring multiple indicators for a pathogen with respect to a number of threshold values, where each indicator may be measured with respect to a one or more threshold values. Further, the absence of a pathogen in the sample may be determined in a similar manner.

The subject, or user (both are used interchangeably herein), can initiate transmission of the first result of the evaluation to the off-site entity 160. Alternatively, the system 100 can automatically transmit the first result of the evaluation to the off-site entity 160 (e.g., utilizing a transmission module 130). The transmission module 130 can transmit the first result of the evaluation via a wired connection, a wireless signal, a digital signal, a Radio Frequency Identification (RFID) tag signal, a communications network, or a wireless tag signal. For instance, the system 100 can include a connection to an interconnection network, such as the Internet or a cellular telephone network. In an embodiment, the system 100 can transmit the first result of the evaluation to an intermediate off-site entity for access by the off-site entity 160. For example, the system 100 can post the result to a website or database, which the off-site entity 160 may then access to retrieve the result. In an embodiment, the system 100 includes a transmission module 130, which can be implemented as a network interface for facilitating connection to the network. The first result can be transmitted to the off-site entity 160 utilizing the network interface. Alternatively, the first result can be provided to the user in the form of a bar code generated based on a result of the sample test, and the user can electronically read or image the bar code and then transmit information generated from reading or imaging the bar code to the off-site entity 160, (e.g., utilizing the network interface, or the like).

Further, the system 100 can include a flash drive (e.g., in the case of a flash drive including an evaluation module 110), and the first result can be transmitted by the flash drive to the off-site entity 160 utilizing a network interface, which can be included with the flash drive, or can be accessible by connecting the flash drive to a personal computer including a network interface. In this instance, the flash drive and/or computer function as a transmission module 130. Further, the first result can be provided to the user in the form of an alphanumeric code, and the user can supply the alphanumeric code (e.g., utilizing a keypad, voice entry, text messaging, or the like) and transmit the alphanumeric code to the off-site entity 160 (e.g., utilizing the network interface, or the like).

The first result of the evaluation can include some or no personal information regarding the subject. All or part of the subject's personal information can be supplied by the subject, a health care provider, a supplier of the system or device. For example, the personal information can include one or more of a sex (gender) of the subject, a name of the subject, a generalization of a name, a biometric identification, a database pointer, a demographic, an age of the subject, a family group of the subject, a location of the subject, or an ethnicity of the subject. In an embodiment, the subject can choose a degree of anonymization of the personal information. For example, the subject can choose to be identified as a member of a household, a resident of an address, a resident of a street, a resident of a neighborhood, a resident of a city, or the like. The personal information may take the form of an alphanumeric referential identification, or a numeric referential identification, for example.

Some types of personal information can be determined by the evaluation module 110 via identifying indicators, such as one or more of an HLA-type, a presence of a genetic marker, a sex hormone level, a Y-chromosomal test, a hormone level, a growth hormone level, or a blood group. For instance, a Y-chromosomal test may be utilized to determine a familial relationship/family group of the subject (e.g., in the case of two males related through their paternal line). In an embodiment, indicators unique to the identity of an individual or a group of individuals may be utilized to verify or memorialize the identity of a subject or a group of subjects, in order to ensure that test results are uniquely associated with specific individuals who provided the samples. In an embodiment, the personal information can be supplied by the subject or a third party. In an embodiment, the personal information can be supplied by the subject or a third party and then provided via the evaluation module 110. The evaluation module 110 can determine personal information utilizing a test strip, a lateral flow test strip, a colorimetric test strip, a transdermal testing device, or a lancet. For example, a lancet may be utilized to collect a blood sample from an individual, which may be analyzed to determine personal information about the individual, such as an identification or demographic of the individual. A transdermal testing device or a lancet may include a microneedle, a microlancet, a microprotrusion, or a microprojection. In an embodiment, specific DNA information obtained from a blood sample acquired from the subject is compared to information stored in a database 166 that includes identifying DNA information about the subject. In an embodiment, the evaluation module 110 may be operably connected to one or more databases, or look-up tables, that can contain, for example, identifying genetic information associated with an individual or a group of individuals. The one or more databases can be associated with the evaluation module 110, or can be external to the evaluation module 110. The one or more databases can be private or publicly available databases.

In an embodiment, the system 100 utilizes software configured to cause or support the transmission of the first result of the evaluation to the off-site entity 160. For example, the software can be configured to execute on at least one of the Internet, a personal communication device, a personal computer, a laptop computer, a palmtop computer, a Personal Digital Assistant (PDA), a mobile telephone, or an image capture device. The software can be provided with an evaluation module 110 or separately from an evaluation module 110. The software can be provided via one or more of a website, a retail outlet, via downloading the software, or via receiving the software via mail. Additionally, the software can be provided by one or more of a government entity or a health clinic.

The transmission module 130 is operably connected to the evaluation module 110. In an embodiment, the transmission module 130 is operably connected to the electronic memory 120. The transmission module 130 is configured to obtain information associated with the first result from the electronic memory 120, and to transmit the first result of the evaluation to an off-site entity 160. In an embodiment, the transmission module 130 is coupled with a network interface module 168 for connecting the transmission module 130 to the off-site entity 160. Further, the network interface module can be coupled with a sensor for sensing network activity on the connection between the transmission module and the off-site entity. In an embodiment, the transmission module 130 is coupled with a scheduling module 170, such as a clock or timer, for scheduling the transmission of the first result of the evaluation. Such transmission can occur at any time, including a time immediately following the provision of the first result to the electronic memory 120, a time of reduced network activity following the provision of the first result to the electronic memory 120, or a scheduled time following the provision of the first result to the electronic memory 120. In an embodiment, the transmission module 130 includes circuitry for receiving information associated with the first result from the electronic memory 120 and circuitry for transmitting the information associated with the first result to an off-site entity 160.

In an embodiment, a system includes a receiving module 162, generally used by the off-site entity 160. The receiving module 162 can be implemented as a network interface, or the like, for receiving the first result of the evaluation from transmission module 130. In an embodiment, the receiving module 162 may be operably connected to one or more databases, or look-up tables, that can contain, for example, information associated with disease states or conditions. In an embodiment, the receiving module is coupled with a database 164 operated by the off-site entity 160. The one or more databases can be associated with the receiving module 162, or can be external to the off-site entity 160. The one or more databases can be private or publicly available databases. In an embodiment, the receiving module 162 includes circuitry for receiving information from the transmission module 130 and circuitry for querying the one or more databases or look-up tables based on the information received from the transmission module 130. In an embodiment, following, and based on, the query of the one or more databases or look-up tables, the receiving module 162 can determine a diagnosis or treatment or provide the results of the database query to the off-site entity 160. In an embodiment, the receiving module 162 includes electronic memory to store the first results of the evaluation, and if desired, personal information or the evaluation module unique identifier.

In an embodiment, the off-site entity 160 determines a second result of the evaluation. The second result of the evaluation can include a diagnosis or treatment regimen based, at least in part, upon the presence or the absence of the at least one pathogen in the sample. The second result of the evaluation can be the first result of the evaluation, or the second result of the evaluation can be an alternate representation of the first result. For example, if the first result includes the presence of a pathogenic antigen in the sample, the second result can include the presence of the pathogenic antigen, or an alternate representation such as an indication of the presence of the pathogen. In an embodiment, the off-site entity 160 communicates the second result of the evaluation to the subject. Such communication can be via the receiving module 162 or by direct communication to the subject via telephone or email, for example. In an embodiment, the receiving module 162 includes circuitry configured to transmit the second result of the evaluation to a reporting module 140. In an embodiment, the reporting module 140 determines the second result of the evaluation, which can include a diagnosis or treatment regimen as previously described. For example, the reporting module 140 can be operably connected to one or more databases, or look-up tables, that can contain, for example, information associated with disease states or conditions. The reporting module 140 can report a second result of the evaluation to the subject. The reporting module 140 can be located proximal to the subject (e.g., apart from the off-site entity 160).

The reporting module 140 can report the second result/diagnosis of the evaluation to the subject after the first result of the evaluation has been queued for transmission. The reporting module 140 can report the second result/diagnosis of the evaluation to the subject before queuing the first result of the evaluation for transmission. In an embodiment, the reporting module 140 can report the second result/diagnosis for several associated subjects. For example, a family unit can utilize one or more evaluation modules 110 for evaluation of a group of family members or the entire family unit. The reporting module 140 can report one or more diagnostic results of the evaluations for the entire family unit (e.g., reporting the presence of a pathogen for a family unit or the presence of a pathogen for individual family members). The reporting module 140 can include display circuitry or other circuitry for communicating information to the subject.

The benefit module 150 is operably coupled to one or more of the evaluation module 110, the reporting module 140, or transmission module 130. The benefit module 150 includes circuitry for receiving information from the evaluation module 110, reporting module 140, or receiving module 162. The benefit module receives information associated with either or both of the first result or the second result of the evaluation and includes circuitry to determine the appropriate benefit to the subject based on the information it receives. For example, the benefit module 150 can initiate the notification of a benefit to the subject via one or more of a postal delivery, an e-mail, an alphanumeric code, a printable document, a notification, a phone call, or a person-to-person contact. In an embodiment, the subject can receive a notification of a benefit via one or more of a wired connection, a wireless signal, a digital signal, a Radio Frequency Identification (RFID) tag signal, a communications network, or a wireless tag signal. The notification of the benefit can be received after the first result of the evaluation has been queued for transmission. The notification of the benefit can be received before queuing the first result of the evaluation for transmission. The notification of the benefit can be received directly or indirectly from the off-site entity. For example, the benefit can be posted to a website or database, which the subject may then access to retrieve the benefit or a notification of the benefit. In an embodiment, the benefit can be determined based, at least in part, on at least one of the first result of the evaluation, a location of the evaluation, a time of the evaluation, or a time of the transmission of the first result of the evaluation. The benefit can be at least partially determined by the off-site entity. In an embodiment, the system includes a location sensing module 172, such as a Global Positioning System (GPS) receiver for determining a location where the evaluation was performed, or a current location of the location sensing module. In an embodiment, the system includes a scheduling module 170, such as a timer or clock for determining a time of the evaluation, or a time when the first result is transmitted. In an embodiment, the benefit can be at least in part determined probabilistically. For example, there may be a 10% chance of receiving a larger/more desirable/more valuable benefit and a 90% chance of receiving a smaller/less desirable/less valuable benefit. Additionally, the benefit can include a benefit for one or more of the subject or a person other than the subject. In an embodiment, a benefit can be determined based on a location of the evaluation in order to target the geographic region of the subject. Alternatively, a more desirable benefit can be provided to an individual in a targeted geographic location, such as a region affected by an epidemic. Moreover, to encourage rapid sampling and evaluation, a more desirable benefit can be provided when an individual utilizes the device 100 within a certain timeframe, such as during the outbreak of an epidemic.

The benefit can include a financial benefit. For example, the financial benefit can include one or more of a monetary benefit, a rebate, a discount, or a coupon. A monetary benefit can be a desirable way to incentivize an individual; however, it should be noted that other benefits can be provided apart from financial benefits; alternatively, a benefit can include a financial component in combination with a non-financial component. In an embodiment, the financial benefit can have a value less than a cost of evaluating the sample, a value substantially equal to a cost of evaluating the sample, or a value greater than a cost of evaluation the sample. In an embodiment, the value of the financial benefit can equal to the cost of acquiring the device 100 in order to offset the cost of purchasing the device. Alternatively, the value of the benefit can be greater than the cost of purchasing the device to further incentivize an individual to utilize the device 100.

The benefit can be associated with providing a therapeutic benefit for the subject. For example, the therapeutic benefit can include one or more of a treatment for the subject, a qualification for treatment for the subject, or a priority status for treatment for the subject. For example, when the result of the evaluation of the sample indicates the presence of a pathogen that is treatable with a particular medication, a benefit can include a notification that the subject qualifies for the medication, which the subject could then have delivered or pick up. Alternatively, when the result of the evaluation of the sample indicates the presence of a pathogen treatable with an injection of a medication, such as a shot available at a public venue, a benefit can include a priority status for receiving the shot.

The benefit can also be associated with providing a preventative benefit. For example, the preventative benefit can include one or more of a vaccine, a qualification for a vaccine, or a priority status for a vaccine. For instance, if the presence or absence of a particular pathogen indicates that the subject can be prone to contracting a particular disease, the benefit can include a vaccination. Alternatively, if the evaluation of the subject indicates a pathogen associated with a particular disease, a relation of the subject (e.g., someone related by household to the subject) can receive a benefit including a vaccine for the indicated disease. In addition, the preventative benefit can include a quarantine protocol. In an embodiment, the preventative benefit can include a notification of health personnel. For example, health personnel can be notified and directed to the residence of the subject to potentially initiate a quarantine protocol.

The benefit can be associated with a diagnostic benefit. For example, the diagnostic benefit can include a recommendation for a test. In some instances, further testing can be required based upon a particular type of pathogen detected, e.g., in order to more accurately determine whether the subject has a particular illness or condition. In an embodiment, the diagnostic benefit can include a means for evaluating a second sample to determine a presence or absence of at least one pathogen in the second sample. For instance, if a pathogen indicates a potential condition, a further test can be provided to the subject based upon the indicated pathogen. In an embodiment, the benefit module 150 may convey an additional benefit if the user initiates a therapeutic benefit/treatment or a preventative benefit.

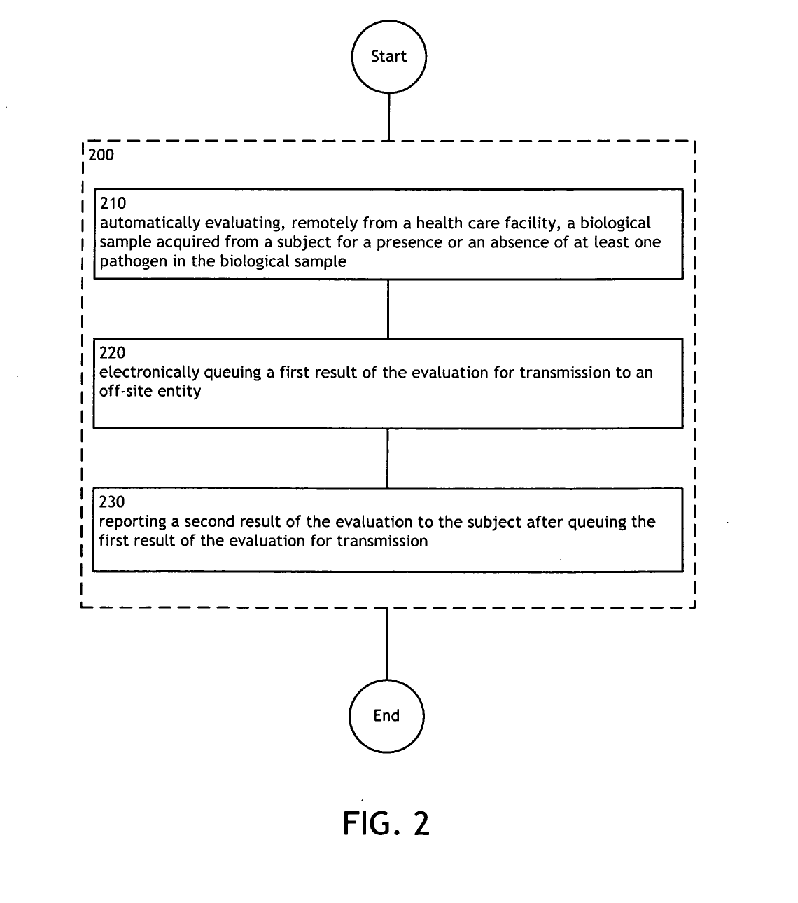

FIG. 2 illustrates an operational flow 200 representing example operations. It should be understood that designations of “start” or “end” in operational flow diagrams herein are not to be construed in a limiting fashion. Such designations are not determinative but are provided as reference points. The illustrated and described processes or methods may be included with other processes or methods that include other steps or features. Nothing herein is intended to convey that no other operations can be performed either or both prior to or following the operations depicted in the figures. In FIG. 2 and in following figures that include various examples of operational flows, discussion and explanation may be provided with respect to the above-described examples of FIG. 1, and/or with respect to other examples and contexts. However, it should be understood that the operational flows can be executed in a number of other environments and contexts, and/or in modified versions of FIG. 1. Also, although the various operational flows are presented in the sequence(s) illustrated, it should be understood that the various operations can be performed in other orders than those which are illustrated, or can be performed concurrently.

After a start operation, the operational flow 200 moves to an operation 210. Operation 210 depicts automatically evaluating, remotely from a health care facility, a biological sample acquired from a subject for a presence or an absence of at least one pathogen in the biological sample.

Then, operation 220 depicts electronically queuing a first result of the evaluation for transmission to an off-site entity.

Then, operation 230 depicts reporting a second result of the evaluation to the subject after queuing the first result of the evaluation for transmission.

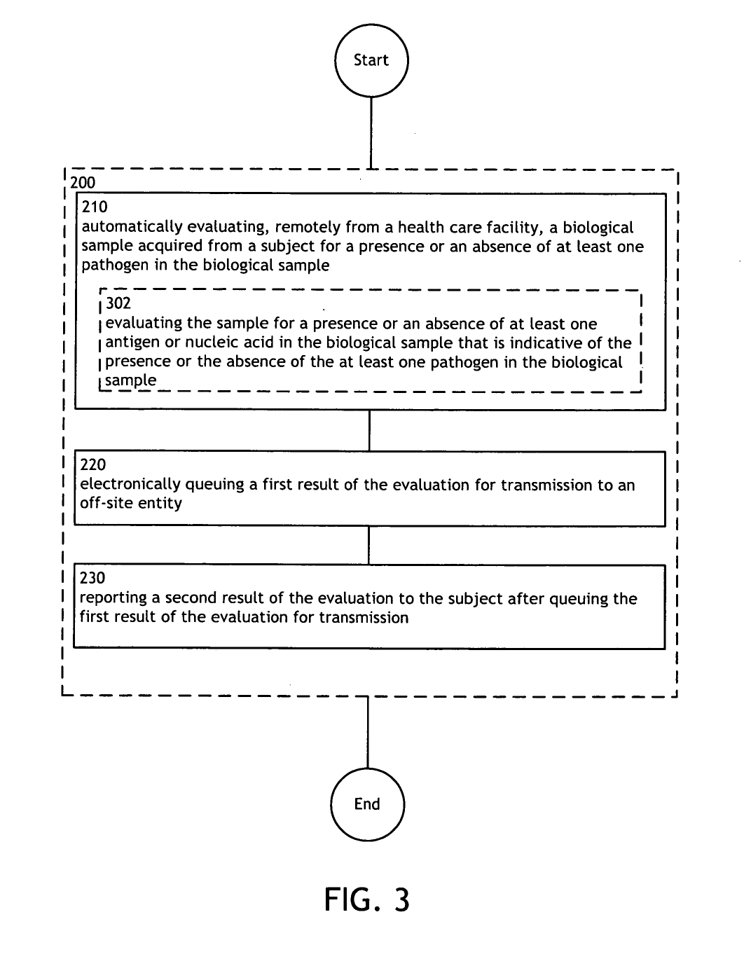

FIG. 3 illustrates alternative embodiments of the example operational flow 200 of FIG. 2. FIG. 3 illustrates example embodiments where the operation 210 can include at least one additional operation. Additional operations can include an operation 302.

The operation 302 illustrates evaluating the sample for a presence or an absence of at least one antigen in the biological sample that is indicative of the presence or the absence of the at least one pathogen in the biological sample.

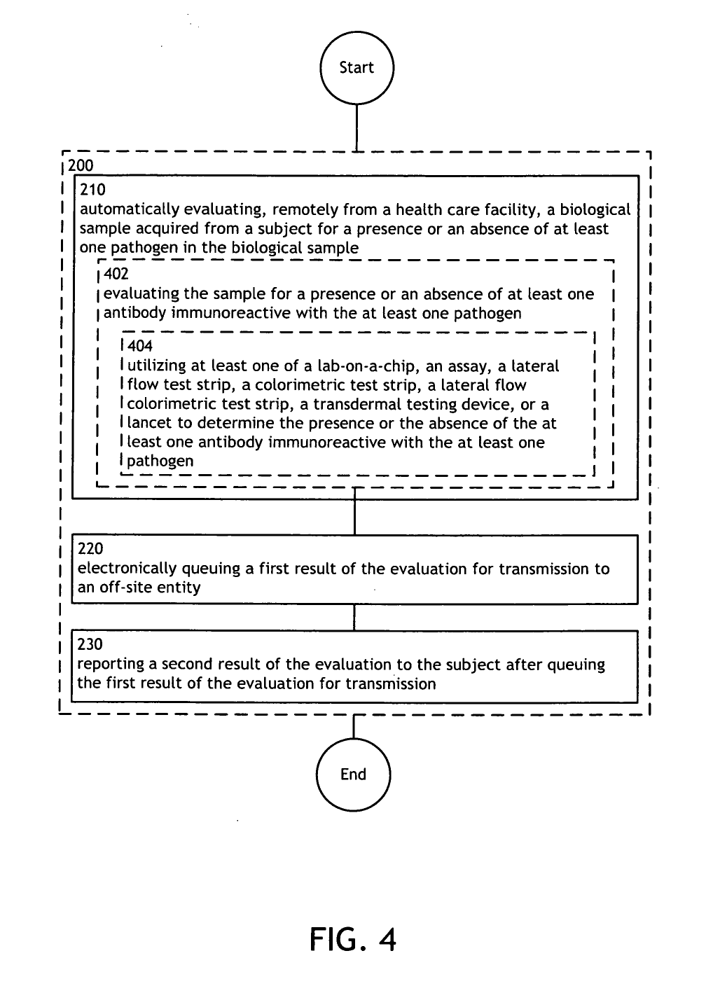

FIG. 4 illustrates alternative embodiments of the example operational flow 200 of FIG. 2. FIG. 4 illustrates example embodiments where the operation 210 can include at least one additional operation. Additional operations can include an operation 402, and/or an operation 404.

The operation 402 illustrates evaluating the sample for a presence or an absence of at least one antibody immunoreactive with the at least one pathogen. Further, the operation 404 illustrates utilizing at least one of a lab-on-a-chip, an assay, a lateral flow test strip, a colorimetric test strip, a lateral flow colorimetric test strip, a transdermal testing device, or a lancet to determine the presence or the absence of the at least one antibody immunoreactive with the at least one pathogen.

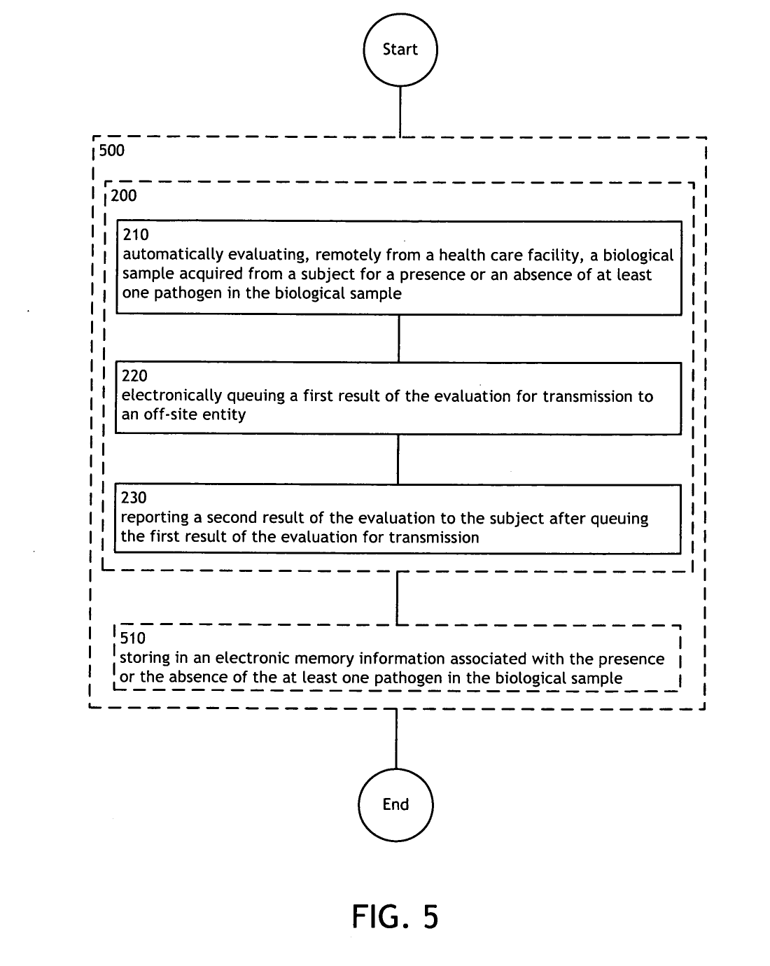

FIG. 5 illustrates an operational flow 500 representing example operations. FIG. 5 illustrates an example embodiment where the example operational flow 200 of FIG. 2 can include at least one additional operation. Additional operations can include an operation 510.

After a start operation, an operation 210, an operation 220, and an operation 230, the operational flow 500 moves to an operation 510. Operation 510 illustrates storing in an electronic memory information associated with the presence or the absence of the at least one pathogen in the biological sample.

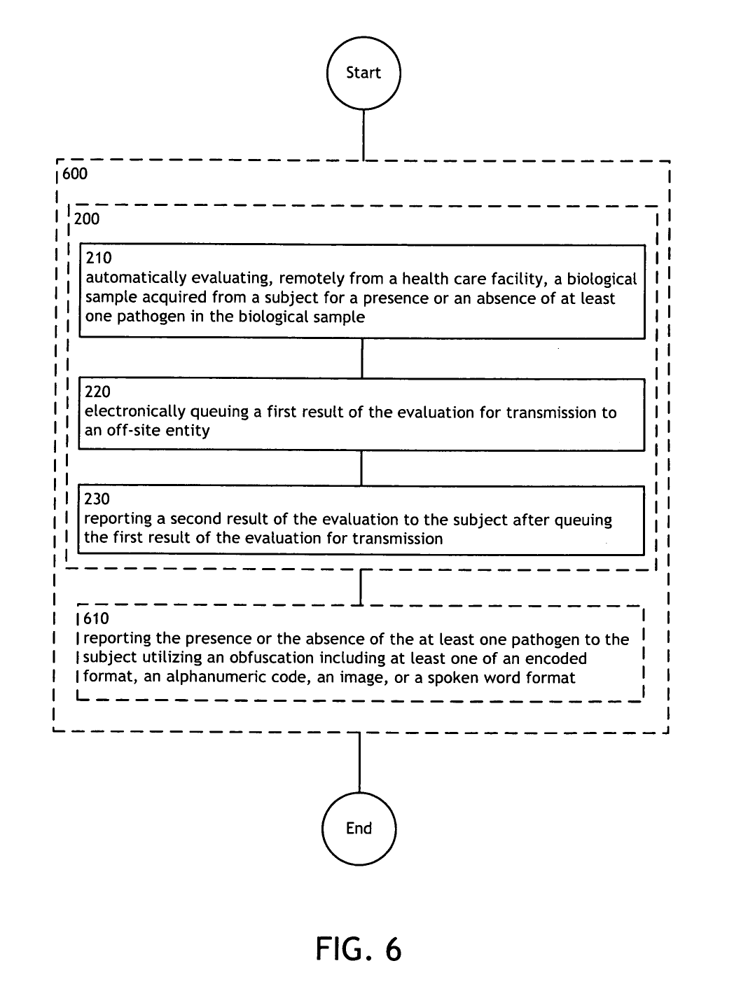

FIG. 6 illustrates an operational flow 600 representing example operations. FIG. 6 illustrates an example embodiment where the example operational flow 200 of FIG. 2 can include at least one additional operation. Additional operations can include an operation 610.

After a start operation, an operation 210, an operation 220, and an operation 230, the operational flow 600 moves to an operation 610. Operation 610 illustrates reporting the presence or the absence of the at least one pathogen to the subject utilizing an obfuscation including at least one of an encoded format, an alphanumeric code, an image, or a spoken word format.

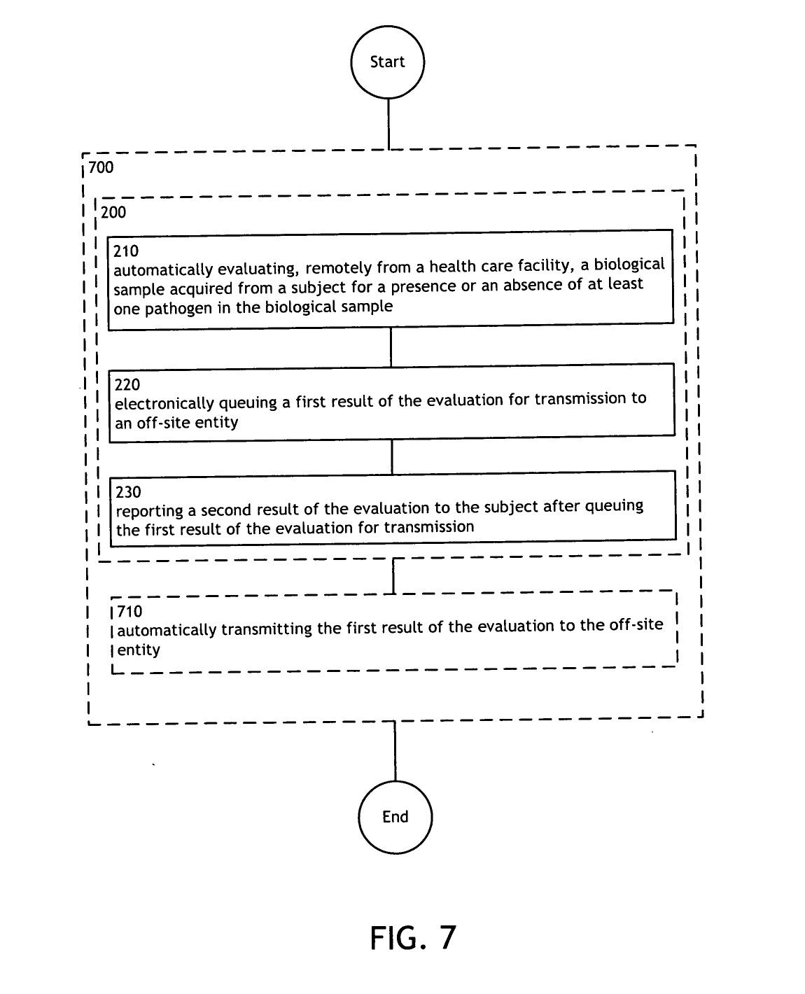

FIG. 7 illustrates an operational flow 700 representing example operations. FIG. 7 illustrates an example embodiment where the example operational flow 200 of FIG. 2 can include at least one additional operation. Additional operations can include an operation 710.

After a start operation, an operation 210, an operation 220, and an operation 230, the operational flow 700 moves to an operation 710. Operation 710 illustrates automatically transmitting the first result of the evaluation to the off-site entity.

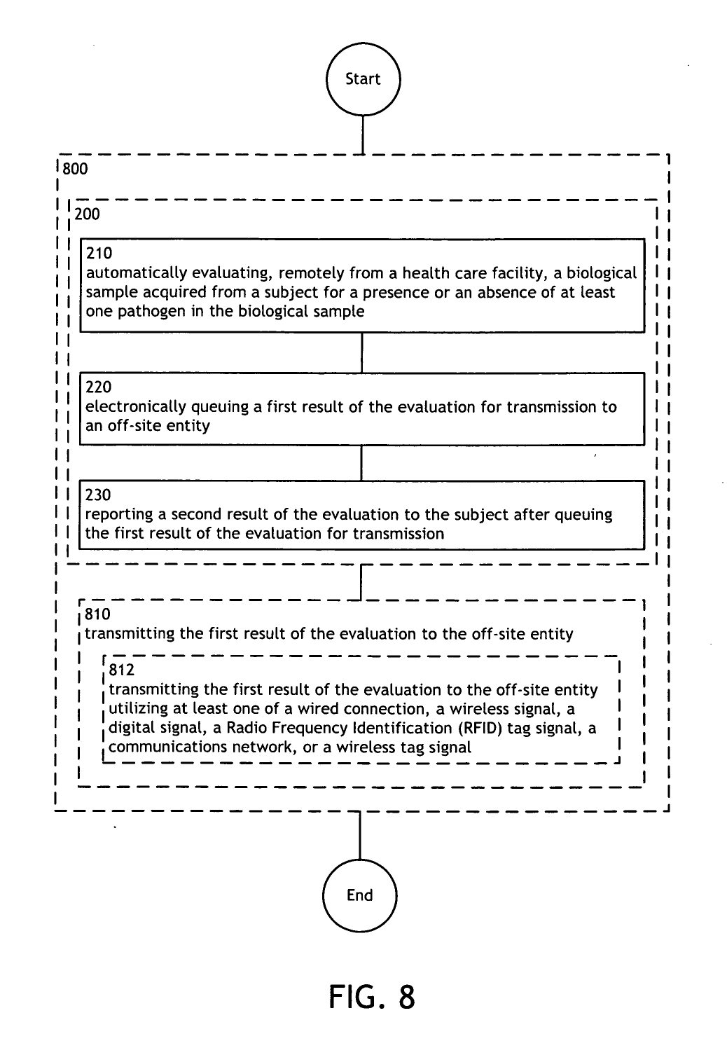

FIG. 8 illustrates an operational flow 800 representing example operations. FIG. 8 illustrates an example embodiment where the example operational flow 200 of FIG. 2 can include at least one additional operation. Additional operations can include an operation 810, and/or an operation 812.

After a start operation, an operation 210, an operation 220, and an operation 230, the operational flow 800 moves to an operation 810. Operation 810 illustrates transmitting the first result of the evaluation to the off-site entity.

The operation 812 illustrates transmitting the first result of the evaluation to the off-site entity utilizing at least one of a wired connection, a wireless signal, a digital signal, a Radio Frequency Identification (RFID) tag signal, a communications network, or a wireless tag signal.

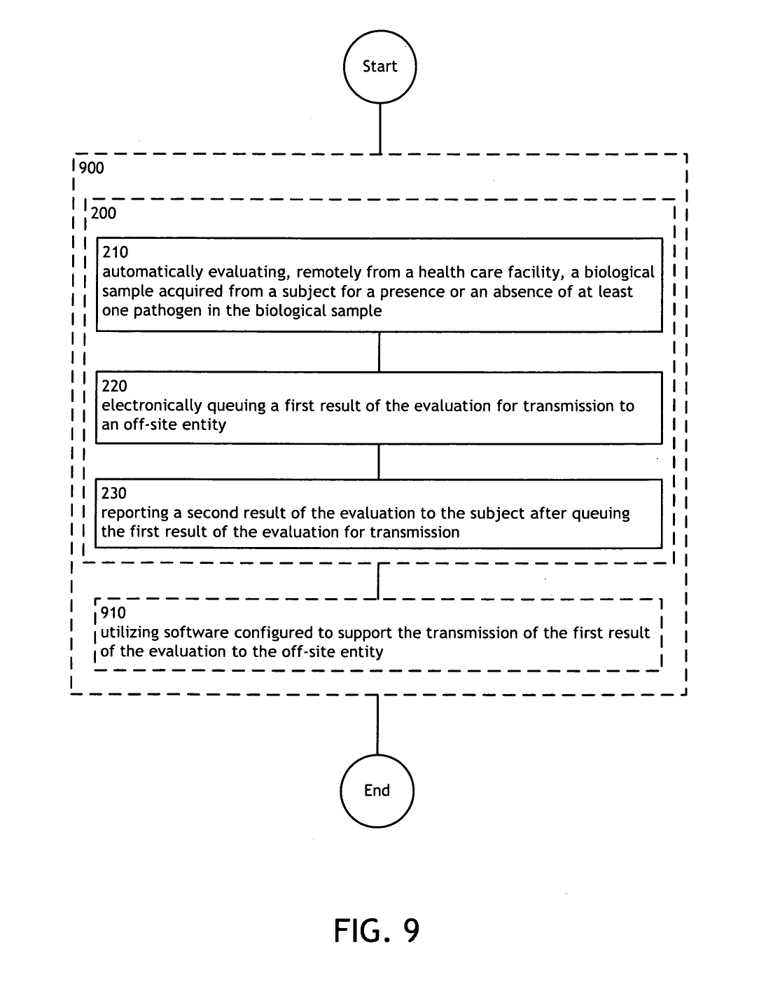

FIG. 9 illustrates an operational flow 900 representing example operations. FIG. 9 illustrates an example embodiment where the example operational flow 200 of FIG. 2 can include at least one additional operation. Additional operations can include an operation 910.

After a start operation, an operation 210, an operation 220, and an operation 230, the operational flow 900 moves to an operation 910. Operation 910 illustrates utilizing software configured to support the transmission of the first result of the evaluation to the off-site entity.

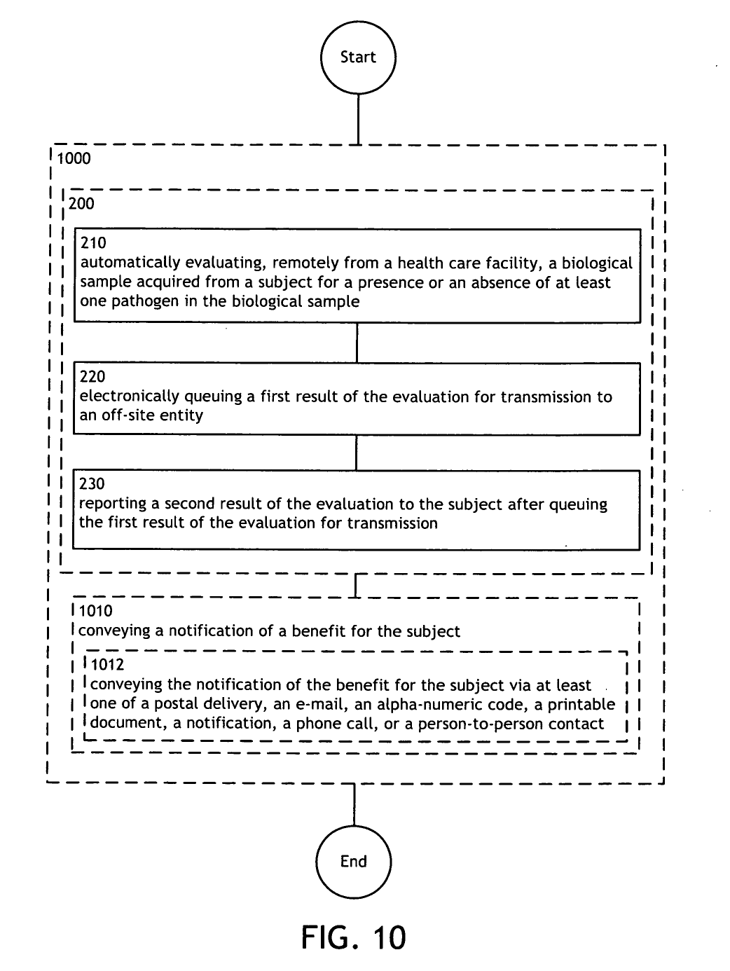

FIG. 10 illustrates an operational flow 1000 representing example operations. FIG. 10 illustrates an example embodiment where the example operational flow 200 of FIG. 2 can include at least one additional operation. Additional operations can include an operation 1010, and/or an operation 1012.

After a start operation, an operation 210, an operation 220, and an operation 230, the operational flow 1000 moves to an operation 1010. Operation 1010 illustrates conveying a notification of a benefit for the subject.

The operation 1012 illustrates conveying the notification of the benefit for the subject via at least one of a postal delivery, an e-mail, an alpha-numeric code, a printable document, a notification, a phone call, or a person-to-person contact.

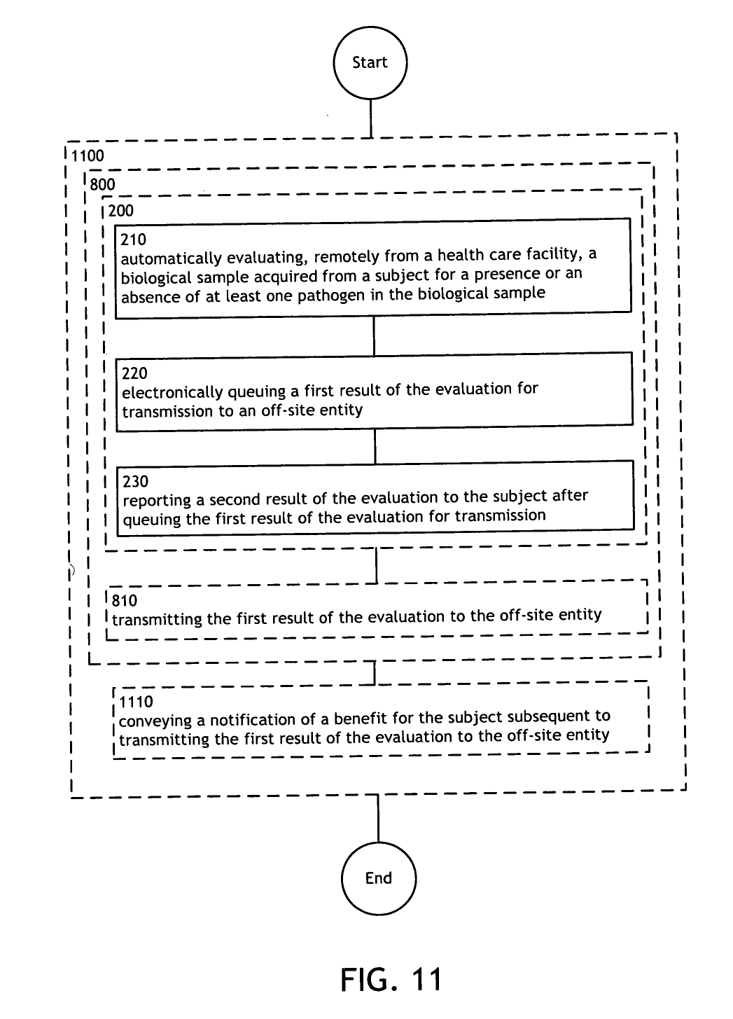

FIG. 11 illustrates alternative embodiments of the example operational flow 800 of FIG. 8. FIG. 11 illustrates example embodiments where the example operational flow 800 of FIG. 8 can include at least one additional operation. Additional operations can include an operation 1110.

The operation 1110 illustrates conveying a notification of a benefit to the subject subsequent to transmitting the first result of the evaluation to the off-site entity.

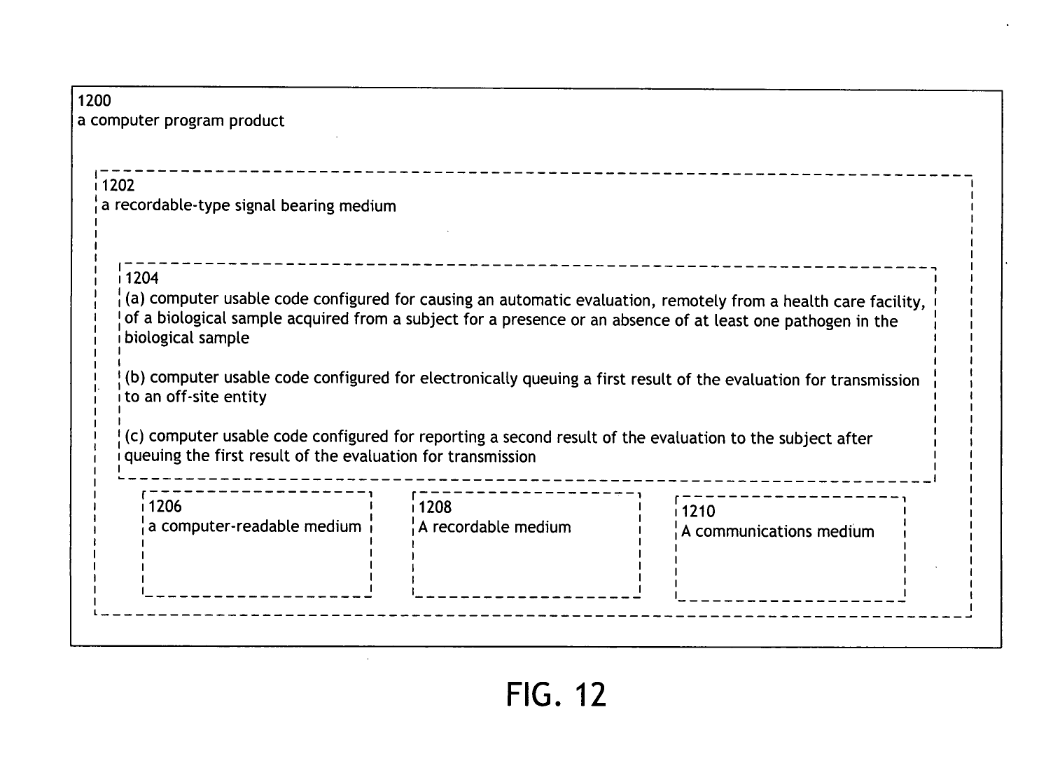

FIG. 12 illustrates a partial view of an example computer program product 1200 that includes a computer program 1204 for executing a computer process on a computing device. An embodiment of the example computer program product 1200 is provided using a recordable-type signal bearing medium 1202, and can include computer usable code configured for causing an automatic evaluation, remotely from a health care facility, of a biological sample acquired from a subject for a presence or an absence of at least one pathogen; computer usable code configured for electronically queuing a first result of the evaluation for transmitting to an off-site entity; and computer usable code configured for reporting a second result of the evaluation to the subject after queuing the first result of the evaluation for transmission. The computer usable code can be, for example, computer executable and/or logic-implemented instructions. In one implementation, the signal-bearing medium 1202 can include a computer-readable medium 1206. In one implementation, the signal bearing medium 1202 can include a recordable medium 1208. In one implementation, the signal bearing medium 1202 can include a communications medium 1210. It should be noted that the second result can be communicated to the subject via a display, or via audio, visual, or other haptic feedback types of communication.

The foregoing detailed description has set forth various embodiments of the devices and/or processes via the use of block diagrams, flowcharts, and/or examples. Insofar as such block diagrams, flowcharts, and/or examples contain one or more functions and/or operations, each function and/or operation within such block diagrams, flowcharts, or examples can be implemented, individually and/or collectively, by a wide range of hardware, software, firmware, or virtually any combination thereof. In one embodiment, several portions of the subject matter described herein can be implemented via Application Specific Integrated Circuits (ASICs), Field Programmable Gate Arrays (FPGAs), digital signal processors (DSPs), or other integrated formats. However, some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a non-transitory signal bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital and/or an analog communication medium (e.g., a fiber optic cable, a waveguide, a wired communications link, a wireless communication link (e.g., transmitter, receiver, transmission logic, reception logic, etc.), etc.).

In a general sense, the various aspects described herein which can be implemented, individually and/or collectively, by a wide range of hardware, software, firmware, and/or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), and/or electrical circuitry forming a communications device (e.g., a modem, communications switch, optical-electrical equipment, etc.). The subject matter described herein can be implemented in an analog or digital fashion or some combination thereof.

The devices and/or processes described herein can be integrated into a data processing system. A data processing system generally includes one or more of a system unit housing, a video display device, memory such as volatile or non-volatile memory, processors such as microprocessors or digital signal processors, computational entities such as operating systems, drivers, graphical user interfaces, and applications programs, one or more interaction devices (e.g., a touch pad, a touch screen, an antenna, etc.), and/or control systems including feedback loops and control motors (e.g., feedback for sensing position and/or velocity; control motors for moving and/or adjusting components and/or quantities). A data processing system can be implemented utilizing suitable commercially available components, such as those typically found in data computing/communication and/or network computing/communication systems.

The herein described components (e.g., operations), devices, objects, and the discussion accompanying them are used as examples for the sake of conceptual clarity and that various configuration modifications are contemplated. Consequently, as used herein, the specific examples set forth and the accompanying discussion are intended to be representative of their more general classes. In general, use of any specific example is intended to be representative of its class, and the non-inclusion of specific components (e.g., operations), devices, and objects should not be taken limiting.

With respect to the use of substantially any plural and/or singular terms herein, the reader can translate from the plural to the singular and/or from the singular to the plural as is appropriate to the context and/or application. The various singular/plural permutations are not expressly set forth herein for sake of clarity.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components, and/or wirelessly interactable, and/or wirelessly interacting components, and/or logically interacting, and/or logically interactable components.

In some instances, one or more components can be referred to herein as “configured to,” “configured by,” “configurable to,” “operable/operative to,” “adapted/adaptable,” “able to,” “conformable/conformed to,” etc. Such terms (e.g. “configured to”) can generally encompass active-state components and/or inactive-state components and/or standby-state components, unless context requires otherwise.

While particular aspects of the subject matter herein have been shown and described, changes and modifications can be made without departing from the subject matter described herein and its broader aspects and, therefore, the appended claims are to encompass within their scope all such changes and modifications as are within the true spirit and scope of the subject matter described herein. In general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). If a specific number of an introduced claim recitation is intended, such an intent will be explicitly recited in the claim, and in the absence of such recitation no such intent is present. For example, as an aid to understanding, the following appended claims can contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). Typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein can generally be performed in any order. Also, although various operational flows are presented in a sequence(s), it should be understood that the various operations can be performed in other orders than those which are illustrated, or can be performed concurrently. Examples of such alternate orderings can include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A computer program product, comprising:
a recordable-type, non-transitory, signal-bearing medium bearing
computer usable code configured for causing an automatic evaluation, remotely from a health care facility, of a biological sample acquired from a subject for a presence or an absence of at least one pathogen in the biological sample;
computer usable code configured for electronically queuing a first result of the evaluation for transmission to an off-site entity; and
computer usable code configured for reporting a second result of the evaluation to the subject after queuing the first result of the evaluation for transmission.

2. The computer program product of claim 1, wherein the biological sample comprises at least one of a fluid, cell, or tissue obtained via a nasal swab, an oral swab, a thoracic swab, a nasal aspirate, an oral aspirate, a thoracic aspirate, a nasal wash, an oral wash, a thoracic wash, a tissue scrape, phlegm, sputum, saliva, urine, or blood collected from the subject.

3. The computer program product of claim 1, wherein automatically evaluating, remotely from a health care facility, a biological sample acquired from a subject for a presence or an absence of at least one pathogen in the biological sample comprises:
evaluating the sample for a presence or an absence of at least one antigen or nucleic acid in the biological sample that is indicative of the presence or the absence of the at least one pathogen in the biological sample.

4. The computer program product of claim 1, wherein automatically evaluating, remotely from a health care facility, a biological sample acquired from a subject for a presence or an absence of at least one pathogen in the biological sample comprises:
evaluating the sample for a presence or an absence of at least one antibody immunoreactive with the at least one pathogen.

5. The computer program product of claim 4, wherein evaluating the sample for a presence or an absence of at least one antibody immunoreactive with the at least one pathogen comprises:
utilizing at least one of a lab-on-a-chip, an assay, a lateral flow test strip, a colorimetric test strip, a lateral flow colorimetric test strip, a transdermal testing device, or a lancet to determine the presence or the absence of the at least one antibody immunoreactive with the at least one pathogen.

6. The computer program product of claim 5, wherein the at least one of the lab-on-a-chip, the assay, the lateral flow test strip, the colorimetric test strip, the lateral flow colorimetric test strip, the transdermal testing device, or the lancet includes a unique identifier.

7. The computer program product of claim 6, wherein the unique identifier includes at least one of a bar code, an RFID tag, or a wireless tag.

8. The computer program product of claim 6, wherein the unique identifier further includes at least one of a lot number, a group assignment, a location of obtainment, a location of reporting, a date of obtainment, a date of reporting, a time elapsed between a date of obtainment and a date of reporting, or an evaluation module type.

9. The computer program product of claim 1, wherein the at least one pathogen includes at least one of a virus, a parasite, a bacterium, a fungus, a toxin, or a toxin-producing pathogen.

10. The computer program product of claim 1, wherein the at least one pathogen includes at least one of an influenza A virus, an influenza A virus subtype, an influenza B virus, or an influenza C virus.

11. The computer program product of claim 1, further comprising:
computer usable code configured for storing in an electronic memory information associated with the presence or the absence of the at least one pathogen in the biological sample.

12. The computer program product of claim 11, wherein the information associated with the presence or the absence of the at least one pathogen in the biological sample is stored in an encoded format.

13. The computer program product of claim 1, wherein the first result of the evaluation includes the presence or the absence of the at least one pathogen in the sample with respect to at least one of a value, a threshold, or a multiplicity of thresholds.

14. The computer program product of claim 1, wherein the first result of the evaluation includes a presence or an absence of at least one pathogenic antigen in the sample.

15. The computer program product of claim 1, wherein the first result of the evaluation includes a presence or an absence in the sample of at least one antibody immunoreactive with the at least one pathogen.

16. The computer program product of claim 1, further comprising:
computer usable code configured for reporting the presence or the absence of the at least one pathogen to the subject utilizing an obfuscation including at least one of an encoded format, an alphanumeric code, an image, or a spoken word format.

17. The computer program product of claim 16, wherein the subject initiates transmission of the obfuscated indication of the presence or the absence of the at least one pathogen to the off-site entity.

18. The computer program product of claim 1, further comprising:
computer usable code configured for automatically transmitting the first result of the evaluation to the off-site entity.

19. The computer program product of claim 1, further comprising:
computer usable code configured for transmitting the first result of the evaluation to the off-site entity.

20. The computer program product of claim 19, wherein transmitting the first result of the evaluation to the off-site entity comprises:
transmitting the first result of the evaluation to the off-site entity utilizing at least one of a wired connection, a wireless signal, a digital signal, a Radio Frequency Identification (RFID) tag signal, a communications network, or a wireless tag signal.

21. The computer program product of claim 19, wherein transmitting the first result of the evaluation to the off-site entity comprises:
transmitting the first result to an intermediate off-site entity for access by the off-site entity.

22. The computer program product of claim 19, wherein transmitting a first result of the evaluation occurs at a first time immediately subsequent to a second time the first result of the evaluation is electronically queued.

23. The computer program product of claim 19, wherein transmitting a first result of the evaluation occurs at a first time of reduced network activity subsequent to a second time the first result of the evaluation is electronically queued.

24. The computer program product of claim 19, wherein transmitting a first result of the evaluation occurs at a scheduled first time subsequent to a second time the first result of the evaluation is electronically queued.

25. The computer program product of claim 19, wherein the second result of the evaluation is determined via the off-site entity.

26. The computer program product of claim 19, wherein the second result of the evaluation comprises the first result.

27. The computer program product of claim 19, wherein the second result of the evaluation comprises an alternate representation of the first result.

28. The computer program product of claim 19, further comprising:
computer usable code configured for conveying a notification of a benefit to the subject subsequent to transmitting the first result of the evaluation to the off-site entity.

29. The computer program product of claim 1, wherein the first result of the evaluation includes personal information regarding the subject.

30. The computer program product of claim 29, wherein the personal information regarding the subject includes at least one of a gender of the subject, a name of the subject, a generalization of a name, a biometric identification, a database pointer, a demographic, an alphanumeric referential identification, a numeric referential identification, an anonymization, an age of the subject, a family group of the subject, a location of the subject, or an ethnicity of the subject.

31. The computer program product of claim 29, wherein the personal information regarding the subject is provided by the subject.

32. The computer program product of claim 29, wherein the personal information regarding the subject is provided by a third party.

33. The computer program product of claim 29, wherein the personal information regarding the subject is determined via at least one of an HLA-type, a presence of a genetic marker, a sex hormone level, a Y-chromosomal test, a hormone level, a growth hormone level, or a blood group.

34. The computer program product of claim 29, wherein the personal information regarding the subject is provided via a lateral flow test strip, a colorimetric test strip, a lateral flow colorimetric test strip, a transdermal testing device, or a lancet.

35. The computer program product of claim 1, further comprising:
computer usable code configured for utilizing software configured to support the transmission of the first result of the evaluation to the off-site entity.

36. The computer program product of claim 35, wherein the software is configured to execute on at least one of the Internet, a personal communication device, a personal computer, a laptop computer, a palmtop computer, a Personal Digital Assistant (PDA), a mobile telephone, an image capture device, or a test strip.

37. The computer program product of claim 1, wherein the second result of the evaluation comprises at least one of a diagnosis or a treatment regimen determined based on information associated with the presence or the absence of the at least one pathogen in the sample.

38. The computer program product of claim 1, wherein the second result of the evaluation is determined via a reporting module.

39. The computer program product of claim 38, wherein the reporting module is associated with the reporting of the second result of the evaluation to the subject.

40. The computer program product of claim 1, further comprising:
computer usable code configured for conveying a notification of a benefit for the subject.

41. The computer program product of claim 40, wherein conveying a notification of a benefit for the subject comprises:
conveying the notification of the benefit for the subject via at least one of a postal delivery, an e-mail, an alpha-numeric code, a printable document, a notification, a phone call, or a person-to-person contact.

42. The computer program product of claim 40, wherein the benefit is determined at least in part based upon the first result of the evaluation.

43. The computer program product of claim 40, wherein the benefit is determined at least in part based upon a location of the evaluation.

44. The computer program product of claim 40, wherein the benefit is determined at least in part based upon a time of the evaluation.

45. The computer program product of claim 40, wherein the benefit is determined at least in part based upon a time of the transmission of the first result.

46. The computer program product of claim 40, wherein the benefit is at least in part determined probabilistically.

47. The computer program product of claim 40, wherein the benefit includes a financial benefit.

48. The computer program product of claim 47, wherein the financial benefit includes at least one of a monetary benefit, a rebate, a discount, or a coupon.

49. The computer program product of claim 47, wherein the financial benefit includes a value less than a cost of evaluating the sample.

50. The computer program product of claim 47, wherein the financial benefit includes a value at least substantially equal to a cost of evaluating the sample.

51. The computer program product of claim 47, wherein the financial benefit includes a value greater than a cost of evaluating the sample.

52. The computer program product of claim 40, wherein the benefit is associated with providing a therapeutic benefit for the subject.

53. The computer program product of claim 52, wherein the therapeutic benefit includes at least one of a treatment for the subject, a qualification for treatment for the subject, or a priority status for treatment for the subject.

54. The computer program product of claim 40, wherein the benefit is associated with providing a preventative benefit.

55. The computer program product of claim 54, wherein the preventative benefit includes at least one of a vaccine, a qualification for a vaccine, or a priority status for a vaccine.

56. The computer program product of claim 54, wherein the preventative benefit includes a quarantine protocol.

57. The computer program product of claim 54, wherein the preventative benefit includes a notification of health personnel.

58. The computer program product of claim 40, wherein the benefit is associated with a diagnostic benefit.

59. The computer program product of claim 58, wherein the diagnostic benefit includes a recommendation for a test.

60. The computer program product of claim 58, wherein the diagnostic benefit includes a means for evaluating a second sample to determine a presence of at least one pathogen in the second sample.

61. The computer program product of claim 40, wherein the benefit includes a benefit for the subject.

62. The computer program product of claim 40, wherein the benefit includes a benefit for a person other than the subject.

63. The computer program product of claim 1, wherein the off-site entity comprises at least one of a medical care provider, a public health entity, or an insurance agency.

64. The computer program product of claim 1, wherein the off-site entity further comprises a receiving module for receiving the first result of the evaluation.

65. The computer program product of claim 1, wherein the off-site entity further comprises a database operably coupled to a receiving module for storing the first result of the evaluation.
